(19) United States
(12) Patent Application Publication
(10) Pub. No.: US 2008/0299207 A1
(43) Pub. Date: Dec. 4, 2008

(54) METHODS AND COMPOSITIONS FOR ADMINISTRATION OF OXYBUTYNIN

(76) Inventor: Michael J. Martin, Chatham, NJ (US)

Correspondence Address: HAYES SOLOWAY P.C., 3450 E. SUNRISE DRIVE, SUITE 140, TUCSON, AZ, 85718, US

(21) Appl. No.: 12/130,903

(22) Filed: May 30, 2008

Related U.S. Application Data
(60) Provisional application No. 60/940,907, filed on May 30, 2007.

(51) Int. Cl. A61K 9/14; A61K 31/216; C07C 69/732; A61P 11/06

(52) U.S. Cl. 424/489; 514/534; 560/58

(57) ABSTRACT

Administration of Oxybutynin in nebulized dry powder form directly to a patient's lungs for treating urinary incontinence or respiratory disease.

### CROSS REFERENCE TO RELATED APPLICATION

This application claims priority from U.S. Provisional Application Ser. No. 60/940,907, filed May 30, 2007, the contents of which are incorporated herein by reference.

### BACKGROUND OF THE INVENTION

1. Field of the Invention

The present invention relates generally to a novel method of administering Oxybutynin, and to novel dosage forms containing Oxybutynin adapted for pulmonary route. The invention will be described in particular in connection with pulmonary delivery of Oxybutynin for prophylactic, therapeutic or ameliorative treatment of incontinence; however, other uses such as pulmonary delivery of Oxybutynin for treatment of respiratory diseases such as asthma and chronic obstructive pulmonary disease (COPD) are also contemplated.

2. Description of the Prior Art

Oxybutynin is a racemic compound of the chemical formula 4-diethylaminobut-2-butynyl phenylcyclohexyl-glycolate:

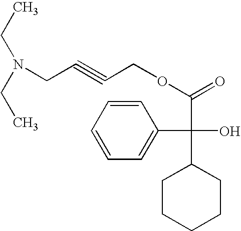

Oxybutynin is an anticholinergic medication that traditionally has been used to treat urge urinary incontinence, urge, frequency and over-active bladder symptoms of incontinence (hereinafter singly and collectively referred to as “urge urinary incontinence”). Oxybutynin acts by decreasing muscle spasms of the bladder. It competitively antagonizes the M1, M2, and M3 subtypes of the muscarinic acetylcholine receptor. It also has direct spasmolytic effects on bladder smooth muscle as a calcium antagonist and local anesthetic, but a concentrations far above those used clinically. It is available orally in generic formulation and as the chloride salt, and as the brand-names Ditropan® and Lyrinel XL®, and as a transdermal patch under the brand-name Oxytrol®.

Oxybutynin currently is administered in oral formulation as a tablet or multiple tablets, or transdermally for treating urge urinary incontinence. However, oral delivery of a therapeutically active amount of Oxybutynin suffers from a number of disadvantages:

(1) Oxybutynin administered in an oral formulation is absorbed from the intestinal track at an undesirably slow and uneven rate that leads to undesirable variations in blood levels and undesirably high dosage rates to achieve a therapeutic response leading to undesirable side effects;

(2) Oxybutynin administered in an oral formulation does not produce desirably high blood levels in a desirably short period of time;

(3) Oxybutynin administered in an oral formation may result in a significant amount not being absorbed because it is being wasted by metabolism or excretion;

(4) Oxybutynin administered in an oral formation is contraindicated for patients with gastrointestinal obstruction disorders because of the risk of urinary retention;

(5) Oxybutynin administered in oral formulation requires chronic dosing with significant side effects, including dry mouth, constipation, blurred vision, drowsiness and dizziness;

(6) Oxybutynin administered in an oral formation is administered as a tablet or multiple tablets which may lack the desirable ease of administration because some people may dislike the swallowing of tablets, or may have difficulty swallowing tablets, or are unable to swallow tablets, or may require a liquid to assist swallowing of tablets; and

(7) Oxybutynin-containing tablets also contain several inactive ingredients, including lactose, corn starch, magnesium silicate, magnesium stearate, and talc which may be considered undesirable because some people may dislike or be allergic to one or more of these inactive ingredients that comprise the Oxybutynin tablets.

Transdermal delivery of Oxybutynin has many of the aforesaid disadvantages. Additionally, some patients suffer skin irritation from transdermal patches.

Thus, there is a need for improved delivery of Oxybutynin, which will provide enhanced bioavailability, minimized variations in blood levels, and achieve more rapid onset of activity, as compared to oral dosage or transdermal dosage forms, while at the same time providing relative ease of administration and reduced side effects compared to current oral and transdermal delivery methods for administering Oxybutynin.

### SUMMARY OF THE INVENTION

The foregoing and other objects of the invention are achieved by providing methods and compositions for pulmonary delivery of Oxybutynin to a mammalian host, particularly a human patient, whereby to provide for rapid absorption of Oxybutynin while avoiding the above and other disadvantages of oral and transdermal administration.

More particularly, it has been discovered that Oxybutynin-containing compositions can be usefully administered to mammals by pulmonary delivery at lower dosage levels to elicit a systemic therapeutic response and provide enhanced bioavailability, minimize variations in blood levels, and achieve more rapid onset of activity, ease of administration, and reduced side effects as compared to conventional oral and transdermal methods of administration, for treating urinary incontinence. Surprising, pulmonary delivery of Oxybutynin provides relief for treating both urinary incontinence and for treating stress urinary incontinence. Being an antispasmodic anticholinergic Oxybutynin also can be expected to provide relief for treating respiratory diseases such as asthma and chronic obstructive pulmonary disease (COPD).

### DETAILED DESCRIPTION OF PREFERRED EMBODIMENTS

As used herein, the term “Oxybutynin” is intended to encompass not only Oxybutynin as an anhydrous powder, but any salt or derivative of Oxybutynin having antispasmodic, anticholinergic activity like Oxybutynin, and which is non-toxic and pharmacologically acceptable, for example, Oxybutynin chloride.

“An effective amount,” as used herein, is an amount of the pharmaceutical composition that is effective for treating urinary incontinence or pulmonary disease, i.e., an amount of Oxybutynin of a defined particle size suitable for absorption in the lungs, that is able to reduce or eliminate the symptoms of urinary and stress incontinence, asthma and COPD.

“A pharmaceutical composition,” as used herein, means a medicament for use in treating a mammal that comprises Oxybutynin in a dry powder form of a defined particle size prepared in a manner that is suitable for pulmonary administration to a mammal. A pharmaceutical composition according to the invention may also, but does not of necessity, include a non-toxic pharmaceutically acceptable carrier.

“A defined particle size,” as used herein, means particles having a size sufficiently small so as to be delivered to the lungs. For optimal delivery to the lungs, the dry powder form of the Oxybutynin preferably should be micronized or spray dried to a maximum powder size of 0.5-10 microns, preferably 1-6 microns.

“A systemically therapeutically effective amount” as used herein will vary with the age, weight and general physical condition of the individual, frequency of dosing, severity of incontinence, and whether urge or stress incontinence, or asthma or COPD is being treated. Generally, for treating urge incontinence, a systemically therapeutically effective amount will comprise the active ingredient in a quantity of from 1 micron to 20 mg/day, preferably 1 to 10 mg/day. The active ingredient may be given once a day. Preferably, however, the active ingredient will be administered in smaller doses two or three or more times a day to maintain more consistent plasma levels. When used for treating stress incontinence, a systematically therapeutically amount will comprise the active ingredient in a quantity of from 1 to 15 mg/kg per dose, preferably 5 to 10 mg/kg per dose, generally administered as a single dose, or as needed. Generally fore treating respiratory diseases, a systemically therapeutically effective amount will comprise the active ingredient in a quantity of from 1 micron to 20 mg/day, preferably 1 to 10 mg/day. The active ingredient may be given once a day. Preferably, however, the active ingredient will be administered in smaller doses two or three or more times a day to maintain more consistent plasma levels.

The dry powder Oxybutynin may then be put into a conventional dry powder inhaler (DPI) in a systemically effective unit dose delivery amount. For treating symptoms of stress urinary incontinence, a dose of Oxybutynin should be taken at the first sign of stress, or upon onset of the first sign of urgency or just prior to anticipated onset of stress, e.g. just before a patient is scheduled to talk in front of an audience. Similarly, for treating symptoms of respiratory distress, a dose of Oxybutynin should be taken at the first sign of respiratory distress. In a preferred embodiment of the invention, the dry powder Oxybutynin is packaged for delivery in a piezo-electronic dry powder inhaler such as described in U.S. Pat. No. 6,026,809.

The dry powder pulmonary delivery of Oxybutynin to the respiratory tract can be used advantageously to treat both urge urinary incontinence and symptoms of stress urinary incontinence. Unlike conventional oral and transdermal delivery of Oxybutynin which require chronic dosing with significant side effects and require hours to reach therapeutically active blood levels, dry powder pulmonary delivery of Oxybutynin permits a patient to enjoy relief at significantly lower doses with concomitant reduction in side effects such as reduced risk of urinary retention. Dry powder pulmonary delivery of Oxybutynin also permits a patient to enjoy relief from symptoms of stress urinary incontinence on an as-needed basis. Similarly, dry powder pulmonary delivery of Oxybutynin permits a patient to enjoy prophylactic relief from symptoms of respiratory distress or on an as needed basis.

The following examples are provided to further illustrate the present invention:

### EXAMPLE 1

Oxybutynin in crystalline form is micronized to a maximum particle size of about 10 microns. The powder is packaged in a dry powder inhaler (DPI) made in accordance with U.S. Pat. No. 6,026,809.

### EXAMPLE 2

Example 1 was repeated, using micronized Oxybutynin chloride of maximum particle size of about 10 microns in place of Oxybutynin.

### CONCLUSION

Delivery of micronized particles of Oxybutynin directly to the lungs, as needed, was found to provide relief to patients suffering from urge urinary incontinence and symptoms of stress urinary incontinence.

While the invention has been described in detail herein in accordance with certain preferred embodiments thereof, many modifications and changes therein may be affected by those skilled in the art. Accordingly, it is intended that the appended claims cover all such modifications and changes as may fall within the spirit and scope of the invention.

We claim:

1. A method for treatment of urinary incontinence comprising delivering directly to a patient's lungs a systemically therapeutically effective amount of Oxybutynin in dry powder form.

2. The method according to claim 1, wherein the Oxybutynin comprises Oxybutynin Chloride.

3. The method according to claim 1, for treating symptoms of stress urinary incontinence.

4. The method according to claim 1, for treating symptoms of urge urinary incontinence.

5. The method according to claim 1, wherein the Oxybutynin is delivered using a dry powder inhaler (DPI).

6. The method according to claim 1, wherein the dry powder inhaler includes a piezo vibrator.

7. The method according to claim 5, wherein the dry powder Oxybutynin is delivered in dry powder form having a maximum powder size of 0.5-10 microns.

8. The method according to claim 6, wherein the dry powder Oxybutynin is delivered in dry powder form having a maximum particle size of 1-6 microns.

9. The method according to claim 3, wherein the therapeutically effective amount is within the range of 1 micron to 20 mg/kg per dose, administered as needed.

10. The method according to claim 9, wherein the therapeutically effective amount is within the range of 1 to 10 mg/kg per dose, administered as needed.

11. The method according to claim 4, wherein the therapeutically effective amount is within the range of 1 micron to 20 mg/day.

12. The method according to claim 11, wherein the therapeutically effective amount is within the range of 1 to 10 mg/day.

13. Oxybutynin in dry powder form having a maximum particle size of 0.5-10 microns.

14. Oxybutynin in dry powder form according to claim 13, wherein the maximum particle size is 1-6 microns.

15. Oxybutynin in dry powder form according to claim 13, in dosage unit form.

16. Oxybutynin in dry powder form according to claim 15, wherein the dose comprises 1 micron to 20 mg/kg.

17. Oxybutynin in dry powder form according to claim 15, wherein the dose comprises 1 to 10 mg/kg.

18. Oxybutynin in dry powder form according to claim 13, in a dry powder inhaler (DPI).

19. Oxybutynin in dry powder form according to claim 1, wherein the dry powder inhaler includes a piezo vibrator.

20. A method for administering Oxybutynin to a mammal suffering from incontinence to treat said incontinence, comprising the steps of:
providing a dry powder inhaler having a chamber containing Oxybutynin in dry powder form; and
coupling a vibrator to said chamber to generate a cloud of dry powder Oxybutynin for delivery to said mammal.

21. A method for treatment of respiratory disease comprising delivering directly to a patient's lungs a systemically therapeutically effective amount of Oxybutynin in dry powder form.

22. The method according to claim 21, wherein the Oxybutynin comprises Oxybutynin Chloride.

23. The method according to claim 21, for treating symptoms of asthma.

24. The method according to claim 21, for treating symptoms of COPD.

25. The method according to claim 21, wherein the Oxybutynin is delivered using a dry powder inhaler (DPI).

26. The method according to claim 21, wherein the dry powder inhaler includes a piezo vibrator.

27. The method according to claim 25, wherein the dry powder Oxybutynin is delivered in dry powder form having a maximum powder size of 0.5-10 microns.

28. The method according to claim 26, wherein the dry powder Oxybutynin is delivered in dry powder form having a maximum particle size of 1-6 microns.

29. The method according to claim 23, wherein the therapeutically effective amount is within the range of 1 micron to 20 mg/kg per dose, administered as needed.

30. The method according to claim 29, wherein the therapeutically effective amount is within the range of 1 to 10 mg/kg per dose, administered as needed.

31. The method according to claim 24, wherein the therapeutically effective amount is within the range of 1 micron to 20 mg/day.

32. The method according to claim 31, wherein the therapeutically effective amount is within the range of 1 to 10 mg/day.

33. A method for administering Oxybutynin to a mammal suffering from pulmonary disease to treat said disease, comprising the steps of:
providing a dry powder inhaler having a chamber containing Oxybutynin in dry powder form; and
coupling a vibrator to said chamber to generate a cloud of dry powder Oxybutynin for delivery to said mammal.
